(19) United States
(12) Patent Application Publication
(10) Pub. No.: US 2019/0392324 A1
(43) Pub. Date: Dec. 26, 2019

(54) Cognitive Analysis and Disambiguation of Electronic Medical Records for Presentation of Pertinent Information for a Medical Treatment Plan

(71) Applicant: International Business Machines Corporation, Armonk, NY (US)

(72) Inventors: Murthy V. Devarakonda, Peekskill, NY (US); Paul C. Tang, Los Altos, CA (US)

(21) Appl. No.: 16/018,301

(22) Filed: Jun. 26, 2018

(51) Int. Cl. G06N 5/00; G06F 17/27; G06F 17/30; G16H 50/20; G16H 20/00; G06F 15/18

(52) U.S. Cl. CPC G06N 5/003; G06F 17/2705; G06F 15/18; G16H 50/20; G16H 20/00; G06F 17/30719

(57) ABSTRACT

A mechanism is provided in a data processing system comprising a processor and a memory, the memory comprising instructions that are executed by the processor to specifically configure the processor to implement a cognitive analysis engine for analysis and disambiguation of electronic medical records for presentation of pertinent information for a medical treatment plan. The cognitive analysis engine receives a medical condition for a current or upcoming interaction with a patient. The cognitive analysis engine receives a medical mental model that emulates the thinking of a medical professional with regard to reviewing a patient electronic medical record (EMR) to identify pertinent information for a medical treatment plan to treat the medical condition. The cognitive analysis engine uses the medical mental model to analyze the EMR for the patient to identify at least one portion of the EMR relevant to the medical treatment plan and to analyze the identified at least one portion of the EMR to extract relevant patient information that is directed to the medical treatment plan for the medical condition. The cognitive analysis engine uses the medical mental model to generate and output a cognitive summary correlating the extracted relevant patient information and the medical treatment plan in association with the medical condition.

### BACKGROUND

The present application relates generally to an improved data processing apparatus and method and more specifically to mechanisms for cognitive analysis and disambiguation of electronic medical records for presentation of pertinent information for a medical treatment plan.

An electronic health record (EHR) or electronic medical record (EMR) is the systematized collection of patient and population electronically-stored health information in a digital format. These records can be shared across different health care settings. Records are shared through network-connected, enterprise-wide information systems or other information networks and exchanges. EMRs may include a range of data, including demographics, medical history, medication and allergies, immunization status, laboratory test results, radiology images, vital signs, personal statistics like age and weight, and billing information.

EMR systems are designed to store data accurately and to capture the state of a patient across time. It eliminates the need to track down a patient's previous paper medical records and assists in ensuring data is accurate and legible. It can reduce risk of data replication as there is only one modifiable file, which means the file is more likely up to date, and decreases risk of lost paperwork. Due to the digital information being searchable and in a single file, EMRs are more effective when extracting medical data for the examination of possible trends and long term changes in a patient. Population-based studies of medical records may also be facilitated by the widespread adoption of EMRs.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising a processor and a memory, the memory comprising instructions that are executed by the processor to specifically configure the processor to implement a cognitive analysis engine for analysis and disambiguation of electronic medical records for presentation of pertinent information for a medical treatment plan. The method comprises receiving, by the cognitive analysis engine executing in the data processing system, a medical condition for a current or upcoming interaction with a patient. The method further comprises receiving, by the cognitive analysis engine, a medical mental model that emulates the thinking of a medical professional with regard to reviewing a patient electronic medical record (EMR) to identify pertinent information for a medical treatment plan to treat the medical condition. The method further comprises analyzing, by the cognitive analysis engine using the medical mental model, the EMR for the patient to identify at least one portion of the EMR relevant to the medical treatment plan. The method further comprises analyzing, by the cognitive analysis engine using the medical mental model, the identified at least one portion of the EMR to extract relevant patient information that is directed to the medical treatment plan for the medical condition. The method further comprises generating and outputting, by the cognitive analysis engine using the medical mental model, a cognitive summary correlating the extracted relevant patient information and the medical treatment plan in association with the medical condition.

In other illustrative embodiments, a computer program product comprising a computer usable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive healthcare system in a computer network;

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 depicts an example medical mental model in accordance with an illustrative embodiment;

FIG. 5 is a block diagram illustrating a mental model instance generation engine in accordance with an illustrative embodiment;

FIG. 6 is a block diagram illustrating a cognitive analysis and disambiguation engine in accordance with an illustrative embodiment;

FIG. 7 is a flowchart of a mechanism for generating a medical mental model instance in accordance with an illustrative embodiment; and

FIG. 8 is a flowchart illustrating operation of a mechanism for cognitive analysis and disambiguation of electronic medical records for presentation of pertinent information for a medical treatment plan in accordance with an illustrative embodiment.

### DETAILED DESCRIPTION

Due to government regulations and advancement in computing technology, many professionals and organizations store patient information in electronic medical records. As the size of these electronic medical records (EMRs) increases, it becomes more difficult for medical professionals to locate and disambiguate information in the EMRs to identify the portions that are of particular relevance to the patient medical conditions being investigated by the medical professional. For example, if the medical professional is treating a patient during an office visit, the medical professional may need to look through the patient's medical history, as stored in the EMR, to identify the particular portions that are relevant to the particular medical issue that the patient is complaining of and/or identify the portions that are of particular importance to previous treatment plans that were applied to the patient. This may be a daunting task, which prior to the implementation of EMRs was a manual task, especially when integration of EMRs from a variety of different sources of information becomes more prolific. That is, when a patient's complied EMRs store information from a variety of different hospitals, pharmacies, emergency clinics, doctors, specialists, etc., it may be difficult to identify what information in these EMRs is of particular relevance to the patient's current medical issues and the particular plan of treatment previously prescribed to the patient. Thus, there is a high likelihood that some pertinent information may be missed. Moreover, the complexity of searching through EMRs to find relevant information leads to frustration on the part of the medical professional.

The illustrative embodiments provide mechanisms that emulate the thinking of a medical professional with regard to reviewing a patient's EMR to identify pertinent information for treating a patient. The mechanisms identify portions of the EMR that are indicative of a previously established plan of treatment for the patient, follow-ups on the plan of treatment, and the specific patient information to be investigated by the medical professional during a current or upcoming interaction with the patient. In some cases, this information may be identified based on a current reason for the patient interaction, e.g., a particular medical condition for which the patient is being seen or for which the patient has scheduled an appointment.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network anchor a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

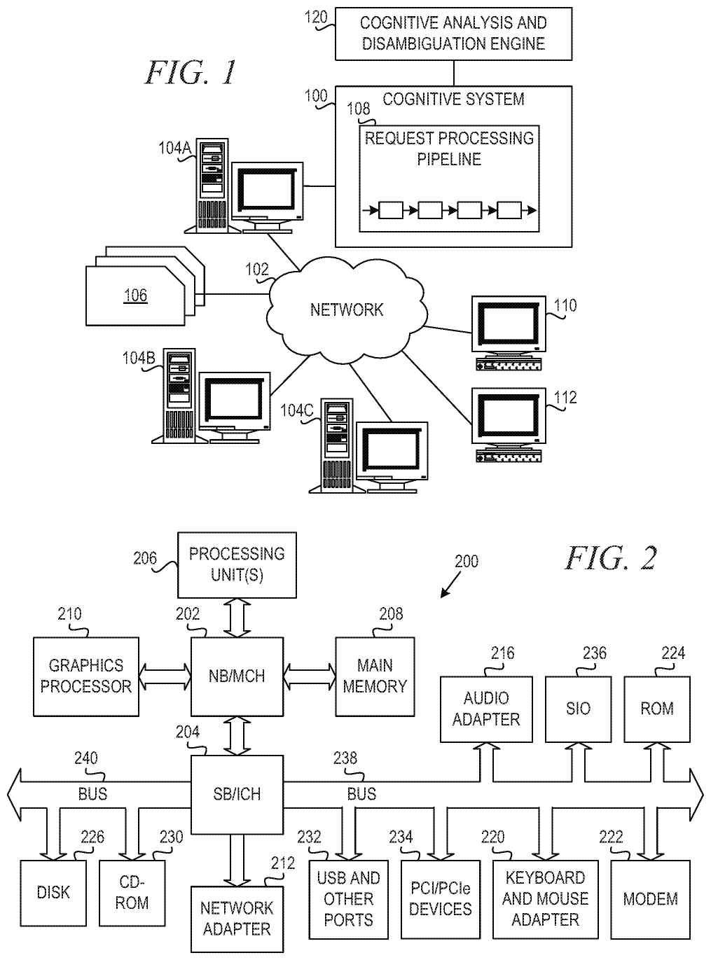

The illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for healthcare applications (also referred to herein as a “healthcare cognitive system”) which implements a request processing pipeline, request processing methodology, and request processing computer program product with which the mechanisms of the illustrative embodiments are implemented. These requests may be provided as structured or unstructured request messages or any other suitable format for requesting an operation to be performed by the healthcare cognitive system. As described in more detail hereafter, the particular healthcare application that is implemented in the cognitive system of the present invention is a healthcare application for cognitive analysis and disambiguation of electronic medical records for presentation of pertinent information for a medical treatment plan.

It should be appreciated that the healthcare cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to a first medical malady domain (e.g., various types of blood diseases) while another request processing pipeline may be trained to answer input requests in another medical malady domain (e.g., various types of cancers). In other cases, for example, the request processing pipelines may be configured to provide different types of cognitive functions or support different types of healthcare applications, such as one request processing pipeline being used for patient diagnosis, another request processing pipeline being configured for cognitive analysis of EMR data, another request processing pipeline being configured for patient monitoring, etc.

Moreover, each request processing pipeline may have its own associated corpus or corpora that it ingests and operate on, e.g., one corpus for blood disease domain documents and another corpus for cancer diagnostics domain related documents in the above examples. In some cases, the request processing pipelines may each operate on the same domain of input requests but may have different configurations, e.g., different annotators or differently trained annotators, such that different analysis and potential answers are generated. The healthcare cognitive system may provide additional logic for routing input requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

As will be discussed in greater detail hereafter, the illustrative embodiments may be integrated in, augment, and extend the functionality of the request processing pipeline and mechanisms of a healthcare cognitive system with regard to an electronic medical record completeness and data quality assessment mechanism.

Thus, it is important to first have an understanding of how cognitive systems in a cognitive system implementing a request processing pipeline is implemented before describing how the mechanisms of the illustrative embodiments are integrated in and augment such cognitive systems and request processing pipeline mechanisms. It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

FIG. 1 depicts a schematic diagram of one illustrative embodiment of a cognitive system 100 implementing a request processing pipeline 108 in a computer network 102. The cognitive system 100 is implemented on one or more computing devices 104A-C (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-C. The network 102 includes multiple computing devices 104A-C, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 may provide cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language request, natural language request for information, natural language request for the performance of a cognitive operation, or the like. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-C on the network 102 include access points for content creators and cognitive system users. Some of the computing devices 104A-C include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and input requests to the cognitive system 100 that are processed based on the content in the corpus or corpora of data 106. In one embodiment, the requests are formed using natural language. The cognitive system 100 parses and interprets the request via a pipeline 108, and provides a response to the cognitive system user, e.g., cognitive system user 110, containing one or more response to the request, results of processing the request, or the like. In some embodiments, the cognitive system 100 provides a response to users in a ranked list of candidate responses while in other illustrative embodiments, the cognitive system 100 provides a single final response or a combination of a final response and ranked listing of other candidate responses.

The cognitive system 100 implements the pipeline 108 which comprises a plurality of stages for processing an input request based on information obtained from the corpus or corpora of data 106. The pipeline 108 generates responses for the input request based on the processing of the input request and the corpus or corpora of data 106.

As noted above, while the input to the cognitive system 100 from a client device may be posed in the form of a natural language request, the illustrative embodiments are not limited to such. Rather, the input request may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for assisting with healthcare based operations. For example, depending upon the particular implementation, the healthcare based operations may comprise patient diagnostics medical practice management systems, personal patient care plan generation and monitoring, or patient electronic medical record (EMR) evaluation for various purposes. Thus, the cognitive system 100 may be a healthcare cognitive system 100 that operates in the medical or healthcare type domains and which may process requests for such healthcare operations via the request processing pipeline 108 input as either structured or unstructured requests, natural language input, or the like.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing a cognitive analysis and disambiguation engine 120 that emulates the thinking of a medical professional with regard to reviewing a patient's EMR to identify pertinent information for treating a patient. Cognitive analysis and disambiguation engine 120 uses knowledge obtained from a variety of different sources of medical information to determine what information is of most importance when investigating a medical condition. Cognitive analysis and disambiguation engine 120 uses a medical mental model that emulates the thinking of a medical professional with regard to reviewing a patient's EMR to identify pertinent information for a medical treatment plan.

Cognitive analysis and disambiguation engine 120 uses natural language processing (NLP) and heuristics to identify instances of such information present in the patient's EMR data. Cognitive analysis and disambiguation engine 120 uses the medical mental model to look for instances of information in the patient's EMR data that deal with what has been done before when treating the patient, what was the planned treatment for the patient, how the medical condition was originally diagnosed, what the goals of the patient and their social determinants were, what motivates the patient to adhere to a treatment plan, etc. Cognitive analysis and disambiguation engine 120 uses the medical mental model to extract meaning from them, with the process being specifically focused on the particular medical condition or reason for the patient's current or upcoming interaction with the medical professional.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and QA system pipeline 108 augmented to include the additional mechanisms of the illustrative embodiments described hereafter.

In the depicted example, data processing system 200 employs a hub architecture including North Bridge and Memory Controller Hub (NB/MCH) 202 and South Bridge and Input/Output (I/O) Controller Hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while Pete does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

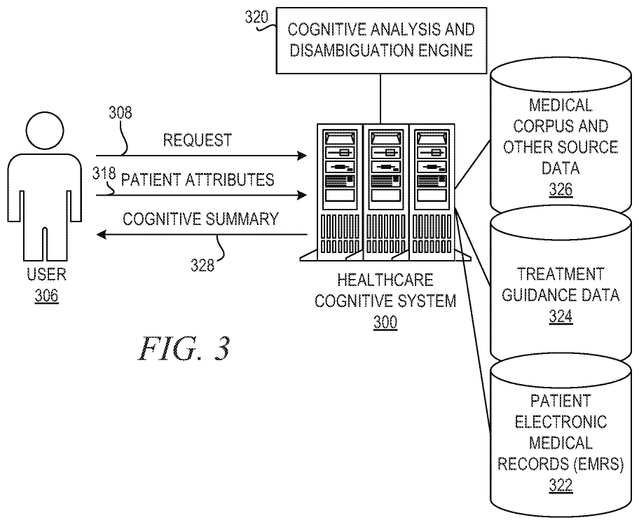

FIG. 3 is an example diagram illustrating an interaction of elements of a healthcare cognitive system in accordance with one illustrative embodiment. The example diagram of FIG. 3 depicts an implementation of a healthcare cognitive system 300 that is configured to provide a cognitive summary of EMR data for patients. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts the user 306 as a human figure, the interactions with user 306 may be performed using computing devices, medical equipment, and/or the like, such that user 306 may in fact be a computing device, e.g., a client computing device. For example, interactions between the user 306 and the healthcare cognitive system 300 will be electronic via a user computing device (not shown), such as a client computing device 110 or 112 in FIG. 1, communicating with the healthcare cognitive system 300 via one or more data communication links and potentially one or more data networks.

As shown in FIG. 3, in accordance with one illustrative embodiment, the user 306 submits a request 308 to the healthcare cognitive system 300, such as via a user interface on a client computing device that is configured to allow users to submit requests to the healthcare cognitive system 300 in a format that the healthcare cognitive system 300 can parse and process. The request 308 may include, or be accompanied with, information identifying patient attributes 318. These patient attributes 318 may include, for example, an identifier of the patient 302 from which patient EMRs 322 for the patient may be retrieved, demographic information about the patient, symptoms, and other pertinent information obtained from responses to requests or information obtained from medical equipment used to monitor or gather data about the condition of the patient. Any information about the patient that may be relevant to a cognitive evaluation of the patient by the healthcare cognitive system 300 may be included in the request 308 and/or patient attributes 318.

The healthcare cognitive system 300 provides a cognitive system that is specifically configured to perform an implementation specific healthcare oriented cognitive operation. In the depicted example, this healthcare oriented cognitive operation is directed to providing a cognitive summary of EMR data 328 to the user 306 to assist the user 306 in treating the patient based on their reported symptoms and other information gathered about the patient. The healthcare cognitive system 300 operates on the request 308 and patient attributes 318 utilizing information gathered from the medical corpus and other source data 326, treatment guidance data 324, and the patient EMRs 322 associated with the patient to generate cognitive summary 328. The cognitive summary 328 may be presented in a ranked ordering with associated supporting evidence, obtained from the patient attributes 318 and data sources 322-326, indicating the reasoning as to why portions of EMR data 322 are being provided.

In accordance with the illustrative embodiments herein, the healthcare cognitive system 300 is augmented to include a cognitive analysis and disambiguation engine 320 that emulates the thinking of a medical professional with regard to reviewing a patient's EMR 322 to identify pertinent information for treating a patient. Cognitive analysis and disambiguation engine 320 uses a medical mental model generated based on knowledge extracted from various sources, such as treatment guidance data 324 and medical corpus and other source data 326. Cognitive analysis and disambiguation engine 320 uses natural language processing (NLP) and heuristics to identify instances of information present in the EMR 322. Cognitive analysis and disambiguation engine 320 uses the medical mental model to identify passages in the patient EMR 322 and extract meaning from them. Cognitive analysis and disambiguation engine 320 uses this information to generate a cognitive summary of a patient's medical record that answers the typical questions about a patient's health condition, consistent with the medical mental model. These include questions about the status of a health condition, the plan of action for that condition, what happened as a result of interventions, and what elements to follow up on by the medical professional during a current or upcoming interaction with the patient.

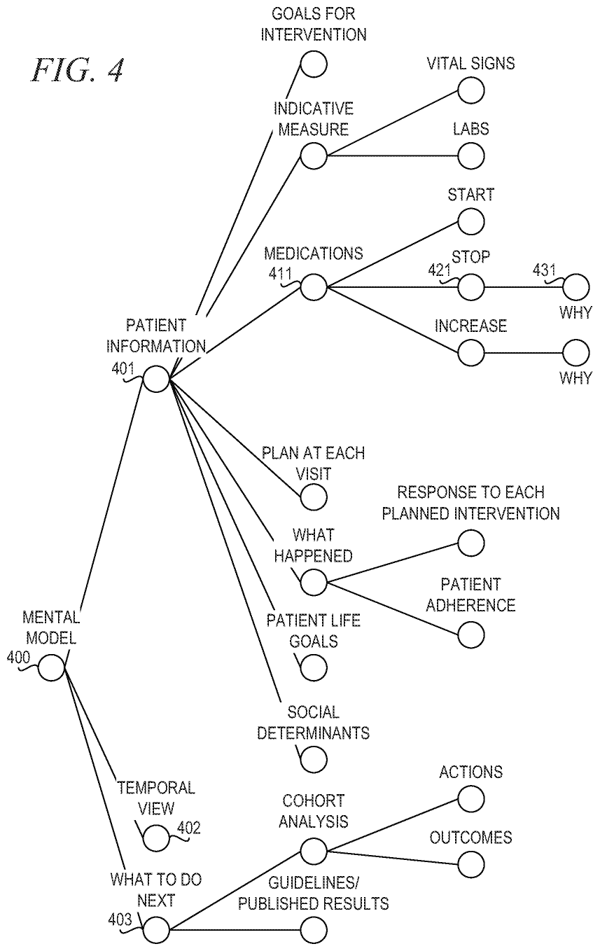

FIG. 4 depicts an example medical mental model in accordance with an illustrative embodiment. The medical mental model is represented as a graph data structure with connected nodes. The medical mental model includes a root node 400 connected to a patient information node 401, temporal view node 402, and what to do next node 403. Patient information node 401 represents patient information the medical professional would look for in the patient EMR. Patient information node 401 is connected to a plurality of child nodes, such as goals for intervention, indicative measure, medications 411, plan at each visit, what happened, patient life goals, and social determinants nodes. These nodes may include words or phrases indicative of data relevant to a medical treatment plan.

In the depicted example, medications node 411 is connected to child nodes, such as start, stop 421, and increase nodes. Stop node 421 is connected to child node, why node 431. In the depicted example, stop node 421 represents words, phrases, or other data to find in the patient EMR when the patient stopped taking a given medication. Why node 431 may represent words, phrases, or other data to find in the patient EMR why the patient stopped taking the given medication.

The medical mental model may also include prototypical questions that a medical professional may ask a healthcare cognitive system about the patient EMR. For example, why model 431 may include the question, “Why did the patient stop taking the medication?” Using the medical mental model, the cognitive analysis and disambiguation engine may submit prototypical questions from the medical mental model to the healthcare cognitive system, which would the return answers to the questions with reference to portions of the patient EMR.

The mental model may be specific to a particular medical professional and to a particular medical condition. Thus, for a particular medical professional, there may be multiple medical mental models, one for each medical condition for which a patient may be treated by the medical professional. For instance, there may be a separate medical mental model for hypertension and diabetes. Furthermore, each medical mental model may be specific to how that medical professional reviews the patient EMR data for the given medical condition.

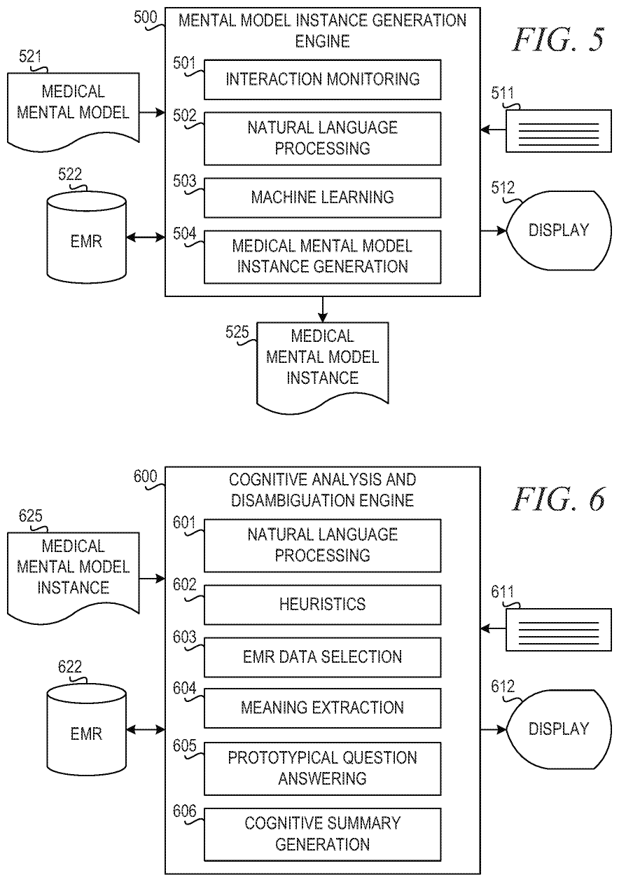

FIG. 5 is a block diagram illustrating a mental model instance generation engine in accordance with an illustrative embodiment. Mental model instance generation engine 500 includes interaction monitoring component 501, natural language processing (NLP) component 502, machine learning component 503, and medical mental model instance generation component 504. Mental model instance generation engine 500 receives medical mental model 521, which is generated using knowledge obtained from a variety of different sources of medical information in consultation with medical professionals. Medical mental model 521 emulates the thinking of a medical professional with regard to reviewing a patient's electronic medical record (EMR) and specifies what information is of most importance when investigating a medical condition.

In one embodiment, medical mental model 521 may be a generic model for all medical professionals in all disciplines. Alternatively, mental model instance generation engine 500 receives one or more medical mental models 521 corresponding to respective medical conditions. For instance, one medical mental model may be for oncologists, while another medical mental model may be for cardiologists, and yet another medical mental model may be for pediatricians.

Each hospital, each medical office, or even each individual medical professional may have a distinct mental model. For example, when treating a patient with heart disease, one medical professional may first look at lab results, while another medical professional may give diet and lifestyle information higher priority. Thus, in accordance with the illustrative embodiment, mental model instance generation engine 500 receives a more general medical mental model 521 and generates a medical mental model instance 525 that is more specific to a given hospital, medical office, or individual medical professional.

Interaction monitoring component 501 monitors user interactions with electronic medical records 522 using input devices 511 and display 512. Input devices 511 may include a keyboard, a mouse, or other known or future input devices. As the user interacts with EMR data 522, interaction monitoring component 501 detects information, such as questions asked by the user, which portions of the EMR the user views, the order in which the user asks questions, the order in which the user views EMR portions, whether the user hovers the mouse cursor over a particular location in the EMR, whether the user zooms in on a portion of an image in the EMR, etc.

Natural language processing (NLP) component 502 performs natural language processing on portions of EMR data 522 being viewed by the medical professional. NLP component 502 may analyze the portions of EMR data 522 to determine key words or phrases that may be relevant to a medical treatment plan for a particular medical condition. This information extracted from the EMR portions viewed by the medical professional may then be used to identify differences between the medical professional's interaction with the EMR data 522 and the general medical mental model 521.

Machine learning component 503 uses machine learning techniques to learn differences between the medical professional interaction with the EMR data 522 and the general medical mental model 521. That is, machine learning component 503 learns when and in what way the medical professional deviates from the medical mental model 521 when interacting with patient EMR data 522.

Medical mental model instance generation component 504 generates medical mental model instance 525 based on the learned differences between the interaction with the patient EMR 522 and the medical mental model 521. Medical mental model instance 525 emulates the thinking of a particular medical office or individual medical professional with regard to reviewing a patient's EMR to identify pertinent information for a medical treatment plan. Medical mental model instance 525 is a combination of usage trends learned from medical professional interaction with a patient EMR, words or phrases indicating a patient treatment plan, patient information to be viewed, steps to take based on the patient information, and prototypical questions to be asked about the patient treatment based on the patient EMR. Medical mental model instance 525 is a data structure that codifies the thinking of a medical professional when reviewing a patient EMR, for a particular medical condition. In one embodiment, medical mental model instance 525 may take the form of a graph data structure. Alternatively, medical mental model instance 525 may be an extensible markup language (XML) document. Other types of data structures, such as linked tables or the like, may be used within the spirit and scope of the illustrative embodiments.

FIG. 6 is a block diagram illustrating a cognitive analysis and disambiguation engine in accordance with an illustrative embodiment. Cognitive analysis and disambiguation engine 600 includes natural language processing component 601, heuristics component 602, EMR data selection component 603, meaning extraction component 604, prototypical question answering component 605, and cognitive summary generation component 606. Cognitive analysis and disambiguation engine 600 organizes and summarizes condition-specific data and context according to the medical mental model instance 625 (inter-related relevant information) for a patient visit.

Natural language processing (NLP) component 601 and heuristics component 602 use NLP and heuristics to identify instances of information present in EMR 620 that is of most importance when investigating a medical condition. Using medical mental model instance 625, natural language processing component 601 and heuristics component 602 look for instances of information in the patient EMR data 620 that deal with what has been done before when treating the patient, what was the planned treatment for the patient, how was the medical condition originally diagnosed, what were the goals of the patient and their social determinants, what motivates the patient to adhere to a treatment plan, etc. EMR data selection component 603 then selects portions of the patient EMR 620 identified using NLP component 601 and heuristics component 602.

Meaning extraction component 604 uses medical mental model instance 625 to identify passages in the patient EMR 620 and extracts meaning from them, with the process being specifically focused on the particular medical condition or reason for the patient's current or upcoming interaction with the medical professional. For example, EMR data selection component 603 may select a portion of the patient EMR data 620 relevant to medications taken by the patient to treat the medical condition, and meaning extraction component 604 may extract from that portion that the patient stopped taking a first medication and started taking a second medication.

Thus, the information in medical mental model instance 625 is then used to identify, for a particular medical condition, portions of the patient EMR that are relevant to the treatment of the medical condition, e.g., for a particular sentence in the patient EMR 620, do features of the sentence match elements of treatment plan for the medical condition? Once identified, meaning extraction component 604 uses medical mental model instance 625 to extract meaning from the identified portions by analyzing the portions to extract relevant patient information that is directed to a treatment plan for the medical condition.

In applying medical mental model instance 625, prototypical question answering component 605 applies prototypical questions for the treatment of the medical condition to extract answers from the identified portions of the patient EMR 620.

Cognitive summary generation component 606 generates a cognitive summary of the patient's EMR 620 that answers the typical questions about a patient's health condition, consistent with the medical mental model instance 625. These include questions about the status of a health condition, the plan of action for that condition, what happened as a result of interventions, and what elements to follow up on by the medical professional during a current or upcoming interaction with the patient. The cognitive summary may then be displayed to the medical professional on display 612.

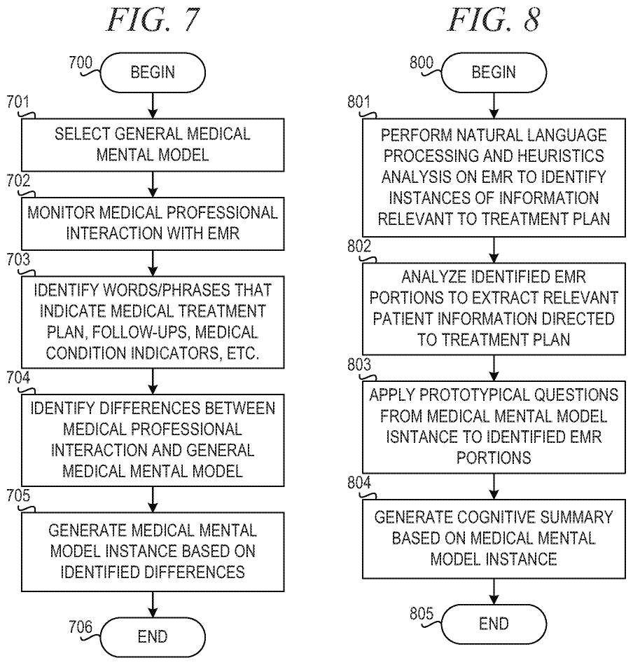

FIG. 7 is a flowchart of a mechanism for generating a medical mental model instance in accordance with an illustrative embodiment. Operation begins (block 700), and the mechanism selects a general medical mental model for the medical professional's specialty or the medical condition being treated (block 701). The mechanism the monitors medical professional interactions with the electronic medical record (EMR) (block 702). The mechanism identifies words or phrases that indicate medical treatment plan, follow-ups, medical condition indicators, etc. (block 703). Then, the mechanism identifies differences between the medical professional's interaction with the patient EMR data and the general medical mental model (block 704). The mechanism generates a medical mental model instance that emulates the thinking of the specific medical office or individual medical professional with regard to reviewing a patient's EMR to identify pertinent information for a medical treatment plan (block 705). Thereafter, operation ends (block 706).

FIG. 8 is a flowchart illustrating operation of a mechanism for cognitive analysis and disambiguation of electronic medical records for presentation of pertinent information for a medical treatment plan in accordance with an illustrative embodiment. Operation begins (block 800), and the mechanism performs natural language processing and heuristics analysis on the patient EMR to identify instances of information relevant to the patient's treatment plan (block 801). The mechanism analyzes the identified EMR portions to extract relevant patient information directed to the treatment plan (block 802). The mechanism then applies prototypical questions from the medical mental model to identify EMR portions (block 803). The mechanism generates a cognitive summary of the EMR based on the medical mental model (block 804). Thereafter, operation ends (block 805).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions that are executed by the processor to specifically configure the processor to implement a cognitive analysis engine for analysis and disambiguation of electronic medical records for presentation of pertinent information for a medical treatment plan, the method comprising:
receiving, by the cognitive analysis engine executing in the data processing system, a medical condition for a current or upcoming interaction with a patient;
receiving, by the cognitive analysis engine, a medical mental model instance that emulates the thinking of a medical professional with regard to reviewing a patient electronic medical record (EMR) to identify pertinent information for a medical treatment plan to treat the medical condition;
analyzing, by the cognitive analysis engine using the medical mental model instance, the EMR for the patient to identify at least one portion of the EMR relevant to the medical treatment plan;
analyzing, by the cognitive analysis engine using the medical mental model instance, the identified at least one portion of the EMR to extract relevant patient information that is directed to the medical treatment plan for the medical condition; and
generating and outputting, by the cognitive analysis engine using the medical mental model instance, a cognitive summary correlating the extracted relevant patient information and the medical treatment plan in association with the medical condition.

2. The method of claim 1, wherein analyzing the EMR for the patient comprises identifying follow-ups on the medical treatment plan in the EMR.

3. The method of claim 1, wherein analyzing the EMR for the patient comprises identifying patient medical condition indicators associated with the medical treatment plan in the EMR.

4. The method of claim 1, wherein the medical mental model instance is specific to the medical condition.

5. The method of claim 1, wherein the medical mental model instance is specific to a medical professional.

6. The method of claim 1, wherein the identified at least one portion of the EMR relevant to the medical treatment plan comprises plans for treatment of the patient, follow-ups with the patient, subjective evaluation of the patient, or objective values.

7. The method of claim 1, wherein analyzing the identified at least one portion of the EMR comprises applying prototypical questions from the medical mental model instance to the identified at least one portion of the EMR.

8. The method of claim 1, further comprising:
selecting a general medical mental model;
monitoring medical professional interaction with the EMR;
identifying differences between the medical professional interaction with the EMR and the general mental model; and
generating a medical mental model instance based on the identified differences.

9. The method of claim 8, wherein identifying the differences comprises using machine learning techniques to learn when and in what way the medical professional deviates from the general medical mental model when interacting with the EMR.

10. The method of claim 1, wherein the medical mental model instance comprises at least one of a graph data structure or an extensible markup language document.

11. A computer program product comprising a computer readable storage medium having a computer readable program stored therein, wherein the computer readable program, when executed on at least one processor of a data processing system, causes the data processing system to implement a cognitive analysis engine for analysis and disambiguation of electronic medical records for presentation of pertinent information for a medical treatment plan, wherein the computer readable program causes the data processing system to:
receive, by the cognitive analysis engine executing in the data processing system, a medical condition for a current or upcoming interaction with a patient;
receive, by the cognitive analysis engine, a medical mental model instance that emulates the thinking of a medical professional with regard to reviewing a patient electronic medical record (EMR) to identify pertinent information for a medical treatment plan to treat the medical condition;
analyze, by the cognitive analysis engine using the medical mental model instance, the EMR for the patient to identify at least one portion of the EMR relevant to the medical treatment plan;
analyze, by the cognitive analysis engine using the medical mental model instance, the identified at least one portion of the EMR to extract relevant patient information that is directed to the medical treatment plan for the medical condition; and
generate and output, by the cognitive analysis engine using the medical mental model instance, a cognitive summary correlating the extracted relevant patient information and the medical treatment plan in association with the medical condition.

12. The computer program product of claim 11, wherein analyzing the EMR for the patient comprises identifying follow-ups on the medical treatment plan in the EMR.

13. The computer program product of claim 11, wherein analyzing the EMR for the patient comprises identifying patient medical condition indicators associated with the medical treatment plan in the EMR.

14. The computer program product of claim 11, wherein the medical mental model instance is specific to the medical condition.

15. The computer program product of claim 1, wherein the medical mental model instance is specific to a medical professional.

16. The computer program product of claim 11, wherein the identified at least one portion of the EMR relevant to the medical treatment plan comprises plans for treatment of the patient, follow-ups with the patient, subjective evaluation of the patient, or objective values.

17. The computer program product of claim 11, wherein analyzing the identified at least one portion of the EMR comprises applying prototypical questions from the medical mental model instance to the identified at least one portion of the EMR.

18. The computer program product of claim 11, wherein the computer readable program causes the data processing system to:
select a general medical mental model;
monitor medical professional interaction with the EMR;
identify differences between the medical professional interaction with the EMR and the general mental model; and
generate the medical mental model instance based on the identified differences.

19. The computer program product of claim 11, wherein the medical mental model instance comprises at least one of a graph data structure or an extensible markup language document.

20. An apparatus comprising:
a processor; and
a memory coupled to the processor, wherein the memory comprises instructions which, when executed by the processor, cause the processor to implement a cognitive analysis engine for analysis and disambiguation of electronic medical records for presentation of pertinent information for a medical treatment plan, wherein the instructions cause the processor to:
receive, by the cognitive analysis engine executing in the data processing system, a medical condition for a current or upcoming interaction with a patient;
receive, by the cognitive analysis engine, a medical mental model instance that emulates the thinking of a medical professional with regard to reviewing a patient electronic medical record (EMR) to identify pertinent information for a medical treatment plan to treat the medical condition;
analyze, by the cognitive analysis engine using the medical mental model instance, the EMR for the patient to identify at least one portion of the EMR relevant to the medical treatment plan;
analyze, by the cognitive analysis engine using the medical mental model instance, the identified at least one portion of the EMR to extract relevant patient information that is directed to the medical treatment plan for the medical condition; and
generate and output, by the cognitive analysis engine using the medical mental model instance, a cognitive summary correlating the extracted relevant patient information and the medical treatment plan in association with the medical condition.
